(19) United States
(12) Patent Application Publication
(10) Pub. No.: US 2004/0249364 A1
(43) Pub. Date: Dec. 9, 2004

(54) Device and method for dispensing medication to tissue lining a body cavity

(76) Inventor: Ilya Kaploun, Toronto (CA)

Correspondence Address: NIXON PEABODY, LLP, 401 9TH STREET, NW, SUITE 900, WASHINGTON, DC, 20004-2128, US

(21) Appl. No.: 10/452,913

(22) Filed: Jun. 3, 2003

(51) Int. Cl. A61K 9/22

(52) U.S. Cl. 604/891.1; 424/424

(57) ABSTRACT

A coil for locally dispensing medication to mammalian tissue is provided. The coil is formed from a length of flexible tubing sealed at opposite ends and containing a medication absorbable into tissue. The tubing walls are semi-permeable to the medication when the coil is in use, but impermeable during storage when the coil is not in use. The permeability of the coil tubing may be controlled either by the application and removal of a water-dissolvable surface coating or envelope, or by the control of an external condition such as temperature. The coil quickly and efficiently delivers a high concentration of medication to tissue in need of treatment, and is particularly useful in the treatment of prostatitis.

### BACKGROUND OF THE INVENTION

1. Field of the Invention

This invention generally relates to medical dispensers, and is specifically concerned with a device and method for dispensing medication to tissue by means of a coil formed from a length of flexible, semi-permeable polymer tubing filled with medication.

2. Description of Related Art

At the present time, most pharmaceuticals are administered orally. Such oral administration is advantageous over the use of injections (i.e. subcutaneous, intramuscular or intravenous), since it is easily conducted by the patient himself, and involves no use of syringes or needles which can cause infections and damage blood vessels and/or nerves if used incorrectly.

While such oral administration of pharmaceuticals works well in a number of medical conditions, there are some medical conditions where such administration is disadvantageous. For example, the conventional treatment of an inflamed prostate gland (known as prostatitis) requires two 500 mg capsules of Cipro® to be orally administered for between 4 and 8 weeks. Even when such a treatment is effective, the applicant has noticed that the administration of such a large amount of antibiotics over such a long time period has a number of deleterious side effects. They include suppression of the immune system of the patient, which can make him more vulnerable to infectious diseases after treatment, as well as the gastrointestinal problems caused by the killing of Acidophilus bacterium and E. coli bacteria in the intestines. Such a treatment is also expensive, costing a patient upwards at least $300.00 for the cost of the antibiotic alone. Finally, such treatment of prostatitis often ends in failure.

The applicant has observed that the failure of such treatment is caused by an insufficient concentration of antibiotic in the prostate area for an insufficient amount of time. Specifically, when oral antibiotics are taken, the concentration of antibiotics in the bloodstream peaks at about four hours from the time they are orally administered and then tapers off to much lower levels several hours later. Effective treatment of prostatitis begins to occur only near the peak concentration, which is too short in duration in most cases to effectively kill the infection responsible for the inflammation. Merely administering larger doses of antibiotics to the patient cannot cure the problem; the severe dosages involved would generate side effects worse than the disease itself.

To solve these problems, devices for locally applying relatively small but concentrated dosages of medications to internal tissues have been developed. An example of such a device is disclosed in Russian Patent No. 1,463,300. Here, a 5 cm long piece of semi-permeable, filamentous polyphenyl amide tubing is first fused closed at one end. This is accomplished by pinching the end of the tubing with heated forceps. Next, the tubing is filled with a medicinal suspension (i.e., an antibiotic and a local anesthetic). The remaining open end of the tube is then also fused shut by heating and pinching in order to form a capsule. Between 5-10 capsules are formed in the aforementioned manner and bundled together. The resulting capsule bundle is then inserted into the rectum of the patient, where they remain until the medicine diffuses outside of the walls of the capsules into the target area, which is the area of the rectum adjacent to the prostate gland.

The use of such semi-permeable capsules to treat prostatitis has shown itself to be far superior to prior art treatments comprising the oral administration of large amounts of antibiotics over a prolonged courses (4-8 weeks). Not only is the success rate of such a capsule-type treatment much higher, it is also much quicker, resulting in a substantial diminution of pain and discomfort within 2-3 hours. It is also much less expensive, since only a fraction of 1 ml of medication is used, versus many grams of antibiotic administered orally in the prior art technique. Finally, because such a small volume of pharmaceuticals is used, virtually no adverse effects have ever been reported.

However, notwithstanding these advantages, the applicant has observed a number of shortcomings associated with the use of such capsules that prevents this technique from realizing its full potential. For example, the rough edges disposed on either end of the 5-10 bundle capsules render it difficult for the patient to self-insert them into a location adjacent to the prostate gland. Instead, a physician or other skilled medical personnel must perform such insertion in order to avoid scratching of the anal orifice and/or mucosal lining of rectum. Additionally, the fabrication of the bundle of capsules is time-consuming and difficult, requiring about 10 to 20 soldering operations before the bundle of capsules is completed. Because the medical suspension begins to diffuse through the walls of the semi-permeable material that forms each of the capsules in about 25-30 minutes, the bundle of capsules has to be custom fabricated immediately prior to insertion into the patient. Finally, the distribution of the medication from the bundle of capsules is accompanied by non-uniformities due to the fact that the outer capsules have a more complete contact with the surrounding tissues than the inner capsules of the bundle.

For all these reasons, there is a need for an improved capsule structure which is more uniformly smooth, and which requires no special skills for insertion such that the patient may easily self-insert it without causing any injury. Ideally, such an improved capsule structure would be easier and faster to fabricate and would be storable for long time periods prior to insertion such that the patient could perform self-insertion at a time and place of his own choosing. Finally, it would be desirable if such an improved capsule structure were accompanied by improved diffusion characteristics such that the medication were more uniformly applied to the inflamed or infected tissue.

### SUMMARY OF THE INVENTION

Generally speaking, the invention is a device and method for dispensing medication to mammalian tissue that overcomes or at least ameliorates all of the aforementioned disadvantages. The device of the invention comprises a coil formed from a length of flexible impermeable tubing which is sealed at opposite ends and which contains a medication absorbable into the tissue of a patient. The flexible tubing is preferably formed from a polymeric material such as filamentous polyphenyl amide. The fact that the coil has only two seals at either end, in combination with its rounded, multi-loop structure, eliminates all potentially sharp edges in the resulting device and facilitates self-insertion. The tubing walls are semi-permeable to the medication to allow diffusion of the same into a patient when the coil is in use, but impermeable to such medication when the coil is not in use.

In one embodiment of the invention, the impermeability of the tubing forming the coil is controlled by way of an ambient condition surrounding the coil, such as temperature or pressure. For example, the viscosity properties of the medication and the porosity of the flexible tubing forming the coil may be selected such that no significant diffusion of medication occurs through the walls of temperatures under about 55° Fahrenheit. In an alternative embodiment, the tubing is rendered impermeable by the application of a selectively removable coating, such as a water-soluble gel. In this embodiment of the invention, the coil may be dipped in water prior to insertion, both to dissolve the gel, and to provide a lubricant that facilitates insertion.

In all embodiments, the coil is formed from a plurality of loops, each of which contacts at least one other loop. In one embodiment of the invention, the loops are all approximately the same diameter, and are secured together by at least two ties, preferably formed from thread made from the same material as the tubing. The resulting coil is ring-shaped and resiliently compressible across its diameter. The fused ends of the tubing forming the coil are preferably located on the inside surface of the coil to avoid the presence of rough edges on the outside of the coil. In an alternative embodiment, the loops are arranged in a spiral configuration, and the resulting coil is substantially flat. A sheet of material for supporting the spiral configuration of loops is adhered to one side of the spiral-type coil. Both types of coils distribute medication more uniformly over their exterior areas, as there is a greater amount of exterior surface area as compared with the bundled capsules of the prior art. Additionally, the smaller amount of tubing seals (i.e., two vs. the 10 to 20 used in the prior art) allows more of the length of the tube to actively distribute medication. In circumstances where two different types of medications are used for treatment (such as an antibiotic and an analgesic), and these medications are not miscible with one another, two coils may be combined and connected together with ties. Alternatively, a single length of tubing may be fused closed in its midsection and the two open ends may be filled with the two different kinds of medication prior to being fused closed.

The first type of coil is particularly applicable to body cavities, such as the rectum and vagina. The second type of coil is particularly applicable to skin wounds or infections, such as chronic skin ulcers, infected post-surgical wounds, or infected burn wounds. When the coil of the first embodiment is used in the vagina of female patients, it may include a drawstring so that it may easily be removed at the termination of treatment. The semi-permeable polymeric tubing used to form the coil preferably has an outer diameter of approximately 1 mm and an internal diameter corresponding to a 21-gauge hypodermic needle to facilitate the introduction of medication into the interior of the tubing.

In the method of the invention, one end of the semi-permeable tubing forming the coil is sealed closed by means of fusion or other plastic soldering technique. The length of tubing (which may be between 40 cm and 100 cm long) is then filled with medication by inserting into the open end of the tube a 21-gauge hypodermic needle. Next, the second end of the tubing is then sealed closed via fusion. The coil is then formed by winding the same into loops, which are either approximately the same size in the case of the first embodiment, or in a spiral configuration in the case of the second embodiment. In the next step of the method, the tubing forming the coil is rendered impermeable to the medication. This can be achieved either by applying a removable coating to the exterior of the tubing prior to filling it with medicinal fluid, or packaging the medicine-filled coil in a water-soluble but medication impermeable envelope, or by applying an ambient condition (such as reduced temperature or increased pressure) to the exterior of the coil which renders it impermeable. Immediately prior to the use of the coil, the tubing forming the coil is rendered semi-permeable to the medication by either removing the coating in the case of the first embodiment, or dissolving the envelope or by changing the exterior condition (i.e., raising the temperature or reducing the pressure) in the case of the second embodiment. The coil is then applied to the tissues of the patient in need of treatment, by either insertion into the appropriate body cavity in the case of the ring-shaped coil, or direct application to the infected or damaged tissues in the case of the spiral-shaped coil. Treatment continues until most or all of the medication diffuses through the walls of the tubing into the surrounding tissue, whereupon the coil is retrieved and discarded.

### BRIEF DESCRIPTION OF THE DRAWINGS

FIG. 1 is a perspective view of a first embodiment of the coil device for dispensing medication of the invention;

FIG. 2 is a top plan view of the coil illustrated in FIG. 1, illustrating in phantom how it flattens out when compressed by surrounding body tissues;

FIG. 3A is a cross-sectional view of the coil as illustrated in FIG. 2 along the line 3-3;

FIG. 3B is a cross-sectional view of the length of tubing forming the coil of the invention;

FIG. 4 are enlarged views of the fusion seals on either end of the coil of the invention;

FIG. 5 is a top plan view of a second embodiment of the invention, illustrating a spiral-shaped coil for dispensing medication to the tissues of a patient; and

FIG. 6 is a cross-sectional view of the coil illustrated in FIG. 5 along the line 6-6.

### DETAILED DESCRIPTION OF PREFERRED EMBODIMENTS

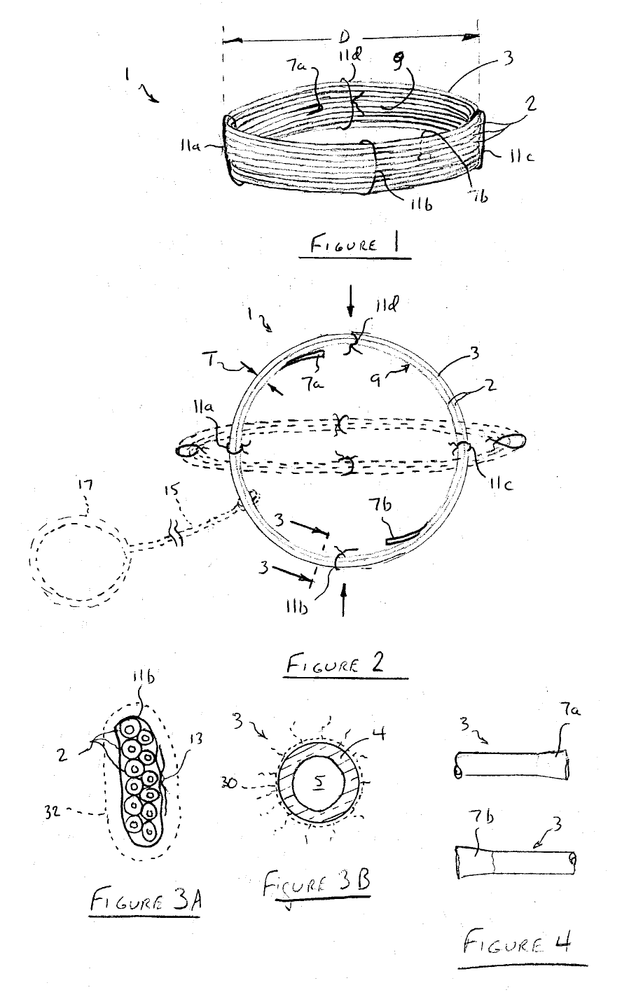

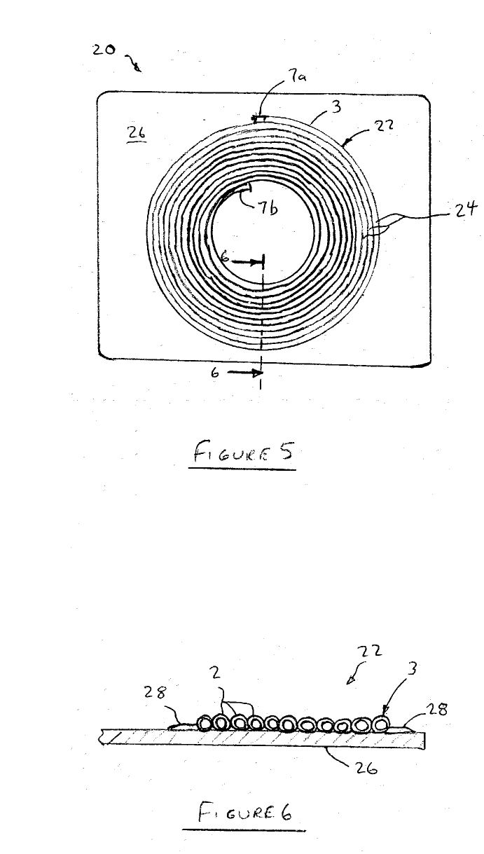

With reference now to FIGS. 1 and 3B, wherein like numerals designate like components throughout all the several figures, the medicinal dispensing device of the invention comprises a ring-shaped coil 1 formed from a plurality of loops 2 of tubing 3 having walls 4 which are semi-permeable to a medication disposed in the interior 5 of the tubing 3. Fusion seals 7a,b on either end of the length of tubing 3 forming the coil 1 serve to contain the medication within the interior 5 of the tubing 3.

In the preferred embodiment, the tubing 3 is formed from filamentous polyphenyl amide tubing having an outer diameter of approximately 1 mm and an inner diameter corresponding to a 21-gauge hypodermic needle to facilitate the filling of the interior 5 of the tubing 3 with a liquid medication. The permeability of the walls 4 of the tubing is matched with the viscosity of the medication in order to provide a desired rate of diffusion which is schematically illustrated in FIG. 3B. In most instances, the permeability of the walls 4 will be set between 15,000-50,000 Daltons. Such tubing may be obtained, for example, from DIC, Inc., located at 3 Protopopovsky by-Street, 129010, Moscow, Russia. Such tubing may also be formed from AMT semi-permeable membranes available from AKZO Nobel having a website at www.electrocoat.com.

With reference to FIGS. 1 and 2, the outer diameter D of the ring-shaped coil 1 of FIG. 1 may be, for example, between about 3.5 and 4 cm, while the internal diameter may be between about 3.4 and 3.9 cm. The height of the coil 1 may be between about 0.8 and 1 cm. The coil 1 may be formed from approximately 8-12 loops 2 of the tubing 3. The fusion seals 7a, 7b are preferably located at the inside surface 9 of the ring-shaped coil 1 so that the exterior surface of the coil 1 presents a uniformly smooth surface with no sharp or irregular edges which can scratch tissue during an insertion process. These fusion seals 7a, 7b may be formed by heating and pinching the ends of the tubing 3, as is best shown in FIG. 4.

With reference in particular to FIGS. 2 and 3A, the loops 2 forming the coil 1 are preferably wound into two layers 10a, 10b and bundled together by ties 11a-11d formed from thread made from the same polyamide material as the tubing 3. The knots 13 formed in the ties 11a-11d are likewise located in the inside surface 9 of the ring-shaped coil 1 in order to avoid the presentation of irregular edges to the exterior surface of the coil 1. The ring-shaped coil 1 may optionally include a pull string 15 that terminates in a ring 17 when it is used to dispense medication to the vaginal area. Such a pull string 15 and ring 17 allows the coil 1 to be easily removed from the vaginal area upon termination of use.

With reference now to FIGS. 2, 3A and 3B, the inherent flexibility of the tubing 3 forming the coil 1 allows it to be easily compressed into the configuration illustrated in phantom in FIG. 2. The ring-shaped coil 1 will naturally assume such a configuration when inserted into a body cavity due to the surrounding pressure of tissues lining such cavities. The compression of the coil 1 into such a flattened configuration advantageously results in the presentation of a large amount of surface area of the coil against surrounding tissue. Such flattening is also accompanied by some splaying of the loops 3 forming the coil 1. Such flattening and splaying promotes a faster and more uniform diffusion of medication from the coil 1 by increasing the amount of surface area contact between the individual loops 3 and the surrounding tissue.

With reference now to FIG. 5, an alternative embodiment 20 of the coil 22 is a spirally wound, single-length of tubing 3 as shown. As was the case with the ring-shaped coil 1, adjacent loops 2 of the flat, spirally-wound coil 22 are in mutual contact with one another in order to concentrate the distribution of the medication contained therein to an adjacent tissue. The spiral windings 24 forming the coil 22 are mounted onto an inert, sterile substrate 26 by means of an adhesive coating 28, as is best seen in FIG. 6. The outer diameter of the spiral shaped coil may be, for example, on the order of 5 to 10 cm. The substrate 26 may be a sterile, biologically neutral mash of the type, which is commercially available for many applications.

The ring-shaped embodiment of the coil 1 may be used to treat the following medical conditions:

Acute and chronic prostatitis (inflammation of the prostate gland) in men;

Acute and chronic cystitis in both men and women;

Post-TURP (Trans Urethral Resection of Prostate); and/or

Post-radical prostatectomy cystitis or low urinary tract symptoms (i.e., frequency, urgency, disuria);

Chronic non-specific proctitis (inflammation of rectal mucosa);

Pelvic Inflammatory Disease in women;

Urethral Syndrome in women;

The flat embodiment of the coil 20 may be used to treat the following conditions:

Chronic skin ulcers;

Infected post surgical wounds;

Infected burn wounds;

Any outside tissue in need of a continuous administration of a high concentration of medication.

In both embodiments of the coil 1 and 20, the walls 4 of the tubing 3 are advantageously rendered impermeable to the medication contained in the interior 5 of the tubing 3 when not in use. This may be accomplished, for example, by the application of an impermeable coating 30 on the outside surface of the tubing walls 4, as is illustrated in FIG. 3B. One example of such a coating would be a water-soluble gel in an instance where the medication contained within the tubing 3 was non-aqueous. Such a coating could be easily removed prior to use by merely dipping the coil in sterile water. The use of such a gel-type coating would have the extra advantage of providing a lubricating film around the coil prior to insertion. Alternatively, such a gel-coated coil could be inserted directly into or applied directly onto body tissues without first dipping the same in sterile water, wherein naturally occurring water-based body fluids would dissolve the coating 30. The impermeable coating need not be confined to water-soluble gels. Any one of a number of other removable coatings may also be used to affect this aspect of the invention. For example, coatings formed from organic compounds, which may be soluble in common antiseptics, such as alcohol, hydrogen peroxide, or bromine-based compounds may also be used. Such non-water dissolvable coating might be used in instances where the medication contained within the tubing 3 is water-based.

Alternatively, the walls 4 of the tubing 3 may be rendered impermeable by packaging the coil 1, 20 in a medication impermeable envelope 32 shown in phantom in FIG. 3A. Like the previously described coating 30, the envelope 32 may be formed from a water-soluble gel that may be first dipped in sterile water to form a lubricant that facilitates insertion, or simply directly inserted into a body cavity, whereupon dissolution of the envelope 32 would occur due to exposure to water-based body fluids.

The walls 4 of the tubing 3 may also be rendered impermeable by an exterior condition such as temperature or pressure. In the case of temperature, the vapor pressure of the medication placed within the tubing 3 might be adjusted such that no significant diffusion occurs through the semi-permeable walls 4 of the tubing 3 in temperatures of 50° Fahrenheit or less. In the case of pressure, the coil 1, 20 might be stored in a pressurized package at room temperature to prevent the medication from diffusing through the walls 4 of the tubing. The pressure inside the package would be adjusted to counter-act the vapor pressure of the medication inside the tubing.

In cases where it is desirable to administer two or more medications simultaneously, and the medications are not miscible with one another or are otherwise chemically incompatible with one another, two coils containing different medications (i.e., an antibiotic an anesthetic) may be manufactured and tied together. For example, the two layers of loops 10a, 10b illustrated in FIG. 3 may be different coils. In such a case, the fused ends of both coils would again be arranged on the interior of the resulting, combined coil to avoid rough exterior edges. The use of two separate coils works particularly well when different tube permeabilities may be required to attain a desired distribution rate of the two medications. Alternatively, a single length of tubing 3 may be pinched and fused in its midsection to close it without dividing it. Different medications may then be used to fill the two open ends of the tubing 3. Advantageously, the closing of the single length of tubing may be performed at a point along the length of the tubing corresponding to a desired ratio of application of the medications. For example, if the physician wishes to apply 3 times more antibiotic than anesthetic, the tubing 3 might be fused or sealed shut at a point 25% of the length from one end of the tubing. This configuration works well when both medications may be distributed by tubing having the same permeability.

The polyphenyl amide semi-permeable tubing 3 used to form either of the coils 1, 20 may contain micro pores of different sizes depending on the molecular weight of the medication contained in the interior 5. While a pore size of 15,000 Daltons is sufficient to allow diffusion of most antibiotics, local anesthetics and other water-soluble substances, a pore size of up to 50,000 Daltons may be necessary for the distribution of larger molecular weight medications such as steroid hormones (e.g., testosterone), oil-based extracts such as Aloe Vera, or proteolytic enzymes such as Chymotrypsin.

The following is a list of exemplary medications which may be distributed via the previously described coil 1, 20:

Antibiotics: Erythromycin, Ampicillin, Amikacin, Ciprofloxacin, Gentamicin, Kanamycin, Cefuroxime, and Clindomycin.

Local anesthetics: Novocain 10%, Lidocaine hydrochloride 2% or 5%.

Anti-inflammatory: Dimethylsulfoxide (Kemsol).

Proteolytic enzymes: Chymopapain, Chymotrypsin, Trypsin, Hyaluronidase.

Antispasmodic: Oxybutynin, Papaverine.

### CLINICAL EXAMPLES OF PATIENTS PARTICIPATING IN TESTS WITH COIL APPLICATIONS

### Example 1

Patient B, 38-year-old man. Diagnosis: Exacerbation of chronic prostatitis.

Patient has been on treatment for this condition for the last 6 months. He has been treated with 6 weeks course of Ciprofloxacin 500 mg twice per day, Naprosyn 250 mg three times per day and Tylenol 500 mg upon more severe pain. Effect of treatment was temporary.

At initial visit patient had constant pain in low abdomen, testicles, and perineum. Pain was getting worse upon physical exertion. Overall level of pain was assessed at 8 (pain assessment scale 0-10). Digital Rectal Examination (DRE): Prostate gland is normal size and firm, but tender on palpation. Microscopic examination of EPS (Expressed Prostatic Secretion): Numerous White Blood Cells (WBCs) in all light fields (×400 magnification). Lecithin granules—low count.

Treatment:

50 cm long coil of polyphenyl amide tubing (15,000 Dalton) filled with suspension of Ampicillin and 5% Lidocaine. Total amount of Ampicillin 500 mg and Lidocaine 1.5 mg were used for one application. Coil was inserted into the rectum and placed over prostate gland for 48 hours.

Patient was contacted 8 hours after application. Level of pain was assessed at 5 (0-10). 20 hrs after procedure—level of pain assessed at 3 (0-10).

46 hrs after procedure: capsules were eliminated with spontaneous bowel movement. Level of pain was assessed at 1 (0-10).

Microscopic examination of EPS (46 hrs after application): 12 WBCs in each light field (×400 magnification).

The same procedure was repeated 2 more times with interval of 4-5 days. Follow-up in 4 months: No complaints, microscopy of EPS-normal.

### Example 2

Patient D, 45-year-old man.

Patient has been suffering from chronic prostatitis for more than 8 years. He has had 3-4 flare-ups of prostatitis annually. His last course of conventional treatment was 3 months ago. He received a total of 30 g of Ciprofloxacin, 4 g of Erythromycin and 90 mg of Terazosine.

At initial visit patient had intermittent dull pain in perineal and sacral areas, urgency, urinary frequency q 1.5 hours, and disuria. Overall level of pain was assessed at 7 (0-10).

DRE: Prostate gland moderately congested, firm and very painful on palpation. Microscopic examination of EPS: Numerous WBCs cover all light fields (×400 magnification), many large clumps of WBCs embedded in mucous.

Treatment:

50 cm coil filled with suspension of Erythromycin, 2% Lidocaine and 0.1 ml of Dimethylsulfoxide. In total patient received 250 mg of Erythromycin, 3 mg of Lidocaine and 0.1 ml of DMSO (Dimethylsulfoxide). Prepared coil was inserted into the rectum and placed over the prostate gland for 24 hours.

8 hours after procedure: pain is assessed at level 5 (0-10).

20 hrs after the procedure: intermittent slight pain in perineal area at level 3 (0-10) and light disuria.

25 hours after procedure—no pain, light urgency, no disuria. The same procedure repeated daily×3.

Microscopy of EPS (after last application): 10-15 WBCs in all light fields, occasional small clumps of WBCs.

Follow-up visit in 4 weeks: Intermittent perineal pain assessed at level 2 (0-10) and frequency q 2-2.5 hours.

The same treatment repeated with 3 coil applications. Follow-up in 4 weeks-patient had no complaints.

### Example 3

Patient C, 40-year-old woman.

She has been suffering with symptoms of urgency, frequency and disuria for more than 10 years. She had numerous treatments for chronic cystitis with rare and short—lasting remissions.

At initial visit patient complained of constant pain in supra pubic area, around urethra. Pain was getting worse during urination. Daytime urinary frequency was less than 1 hour. Nighttime urinary frequency was 3-5 times per night. Overall level of pain was assessed at 7 (0-10).

Laboratory tests: Complete Blood Count, urinalysis and urine C&S were normal. Cystoscopy: showed some inflammation in mucosal lining of bladder neck. Cystomanometry: hyperreflexic and hypertonic bladder. Intravenous Pyelography (IVP)—normal.

Urethral and cervical orifices swabs for culture were positive for Streptococcus haemoliticus.

Vaginal 4-point swabs for culture revealed Staphylococcus aureus in 2 swabs.

Treatment:

50 cm coils were filled with suspension of Cefuroxime, 5% Lidocaine and 0.2 ml of DMSO. Total dose of medications used: Cefuroxime—250 mg, Lidocaine—5 mg, DMSO—0.2 ml. Coil was manually inserted into anterior fornix of vagina for 48 hours.

In 10 hours patient noticed diminishing of pain. The level of pain that time was assessed at 5 (0-10) Nighttime urination was 2 times. Forty-eight hours after the application, a nurse manually removed the coil. In 3 days the patient had no complaints.

In 4 days after first application the procedure was repeated again with the same medicinal combination.

Follow-up in 3 months: patient had no complaints.

### Example 4

Patient D, 62-year-old woman.

She had a right buttock incised abscess. The post incision wound measured 5×2.5×3 cm. The wound was treated daily with conventional dressings for 6 weeks, but was healing very poorly due to patient's long time insulin dependant diabetes.

On examination the post incision wound was measuring 5 cm long, 2 cm wide and 2 cm deep wound was lacking granulations and the walls were covered with necrotic tissues and there was a moderate purulent discharge.

Treatment:

The wound was initially rinsed with NS (normal saline) Sterilized 20 cm long coil was filled with suspension of 1,000 mg of Ampicillin and 0.3 ml of 2% Lidocaine. Coil was placed into the wound by using sterile technique and covered with sterile gauze soaked in NS. The same type of dressing was repeated for 5 days.

On 6^{th} day 40 cm long sterile coil was filled with 150 units of Chymotrypsin and 0.3 ml of 2% Lidocaine. Wound was covered with gauze soaked in NS. The same type of dressing was done 2 times per day for 4 days and then replaced by first coil combination for another 7 days. By the 17^{th} day the wound was cleared from necrotic tissue, there was a small serous discharge and the walls of the wound were covered with fresh pink granulations. Conventional dressings were resumed at this point and wound completely healed in 10 days after that.

The following advantages in treatment are realized by the coil 1, 20 of the invention:

Long-term maintenance of optimal drug concentration in area of disease.

Gradual increase in concentration of medicines in the area of their effect on rectal mucosa (Gradual diffusion through fine porous membrane of coil). It prevents possible adverse effect of medications on rectal mucosa (very unlikely) (<1%).

Significant decrease in dose of used medications (total dose is 5-10 folds less compared to conventional treatment).

Local concept of treatment.

No adverse reactions reported.

Low cost.

Simple procedure, accessible on all levels of health care.

Can be self-administered or performed by either a physician or a nurse.

While the invention has been described in terms of two preferred embodiments, persons of skill in the art will recognize that many modifications, variations and additions may be made with respect to these preferred embodiments. All such variations, modifications and additions are within the scope of this invention, which is limited only by the appended claims and all equivalents to the recited elements of these claims.

What is claimed is:

1. A device for dispensing medication to mammalian tissue, comprising: A coil formed from a length of flexible tubing, said tubing being sealed at opposite ends and having an interior containing a medication absorbable into said mammalian tissue, and tubing walls that are semi-permeable to said medication when said coil is in use to treat said tissue but which are impermeable to said medication when said coil is not in use.

2. The device for dispensing medication to mammalian tissue according to claim 1, wherein said tubing is rendered impermeable to said medication upon the selective control of a condition that is external to said coil.

3. The device for dispensing medication to mammalian tissue according to claim 2, wherein said external condition is temperature.

4. The device for dispensing medication to mammalian tissue according to claim 2, wherein said external condition is pressure.

5. The device for dispensing medication to mammalian tissue according to claim 1, wherein said tubing is rendered impermeable by the application of a selectively removable coating.

6. The device for dispensing medication to mammalian tissue according to claim 1, further comprising a medication impermeable, water soluble envelope that covers and seals said coil, said tubing walls being impermeable to said medication until said envelope is dissolved.

7. The device for dispensing medication to mammalian tissue according to claim 6, wherein said envelope functions as a lubricant when in a state of dissolution.

8. The device for dispensing medication to mammalian tissue according to claim 1, wherein said coil is formed from a plurality of loops, each of which contacts at least one other loop.

9. The device for dispensing medication to mammalian tissue according to claim 8, wherein said loops are approximately the same diameter secured together by at least two ties to form a ring-shaped coil, and said coil is resiliently compressible across its diameter.

10. The device for dispensing medication to mammalian tissue according to claim 8, wherein said loops are arranged in a spiral configuration, and said coil is substantially flat.

11. The device for dispensing medication to mammalian tissue according to claim 10, further comprising a sheet of material for supporting said spiral configuration of loops.

12. The device for dispensing medication to mammalian tissue according to claim 1, wherein said tubing is formed from a filamentous synthetic material.

13. The device for dispensing medication to mammalian tissue according to claim 1, further comprising a second coil of flexible tubing being sealed at opposite ends to contain a different medication from said first coil and being joined to said first coil.

14. The device for dispensing medication to mammalian tissue according to claim 1, further comprising a drawstring attached to said coil at one end for removing said coil from an area of tissue.

15. A device for dispensing medication to tissue lining a body cavity, comprising:
A flexible coil formed from multiple, mutually contacting loops of a length of flexible tubing, and tubing being sealed at opposite ends and having an interior containing a medication absorbable into said mammalian tissue, and tubing walls that are semi-permeable to said medication during use but which are impermeable to said medication when said coil is not in use.

16. A device for dispensing medication to tissue lining a body cavity according to claim 15, wherein said tubing includes an exterior dissolvable coating for rendering said tubing walls impermeable until use.

17. A device for dispensing medication to tissue lining a body cavity according to claim 15, wherein said coating is water soluble.

18. A device for dispensing medication to tissue lining a body cavity according to claim 15, wherein said tubing is rendered impermeable to said medication upon the selective control of an exterior condition that is external to said coil and different from conditions during use.

19. A device for dispensing medication to tissue lining a body cavity according to claim 18, wherein said external condition is maintaining said coil at a temperature less than about 60° F.

20. A device for dispensing medication to tissue lining a body cavity according to claim 18, wherein said external condition is maintaining said coil at an elevated pressure.

21. A device for dispensing medication to tissue lining a body cavity according to claim 15, wherein said tubing is synthetic tubing having a porosity of between about 15,000 to 50,000 Daltons.

22. A device for dispensing medication to tissue lining a body cavity according to claim 15, wherein said tubing is formed from a polymeric material.

23. A device for dispensing medication to tissue lining a body cavity according to claim 15, wherein said tubing has an exterior diameter of between about 0.5 and 2.0 mm, and said length of tubing is between about 0.25 and 0.75 m.

24. A device for dispensing medication to tissue lining a body cavity according to claim 15, wherein said tubing contains between about 0.05 cc and 0.40 cc of medication.

25. A device for dispensing medication to tissue lining a body cavity according to claim 15, wherein said loops are secured together by at least two ties formed from thread made of the same material as said tubing.

26. A method for locally dispensing medication to mammalian tissue, comprising the steps of:
filling a length of flexible tubing with medication, said tubing being formed of a material that is semi-permeable to said medication;
sealing said length of tubing to contain said medication;
winding said tubing into a coil, and rendering said tubing of said coil impermeable to said medication until such time that said coil is used to treat tissue.

27. A method for locally dispensing medication to mammalian tissue according to claim 26, wherein said flexible tubing is coated with a removable coating to render it impermeable to said medication, and said coating is removed immediately before said coil is used to treat tissue.

28. A method for locally dispending medication to mammalian tissue according to claim 26, wherein said coil is packaged within a medication insoluble envelope prior to use to render said tubing non-permeable to said medication, said envelope being water soluble such that said tubing is rendered permeable upon an aqueous dissolution of said envelope.

29. A method for locally dispensing medication to mammalian tissue according to claim 27, wherein tubing is rendered impermeable by changing an external condition of said coil.

30. A method for locally dispensing medication to mammalian tissue according to claim 29, wherein said tubing is rendered impermeable by lowering an ambient temperature surrounding said coil to at least 55° F.

31. A method for locally dispensing medication to mammalian tissue according to claim 26, wherein said tubing is wound into multiple loops of approximately the same diameter to form a ring-shaped coil.

32. A method for locally dispensing medication to mammalian tissue according to claim 26, wherein said tubing is spirally wound to form a flat coil.

33. A method for locally dispensing medication to mammalian tissue according to claim 32, further comprising the step of adhering a piece of sheet material to one side of said flat coil to support the same.
